(19) United States
(12) Patent Application Publication
(10) Pub. No.: US 2024/0197468 A1
(43) Pub. Date: Jun. 20, 2024

(54) ACCOMMODATING INTRAOCULAR LENSES AND ASSOCIATED METHODS

(71) Applicant: Shifamed Holdings, LLC, Campbell, CA (US)

(72) Inventors: Juan Diego Perea, San Jose, CA (US); Cooper Burton, San Jose, CA (US)

(21) Appl. No.: 18/555,777

(22) PCT Filed: Apr. 22, 2022
(86) PCT No.: PCT/US2022/025887

§ 371 Date: Oct. 17, 2023

Related U.S. Application Data
(60) Provisional application No. 63/178,275, filed on Apr. 22, 2021.

(51) Int. Cl. A61F 2/16

(52) U.S. Cl. CPC A61F 2/16; A61F 2002/169; A61F 2002/16902; A61F 2220/0033; A61F 2240/004; A61F 2250/0063; A61F 2250/0087; A61F 2250/0097

(57) ABSTRACT

An accommodating intraocular lens (AIOL) can include a base lens and a removable lens. The base lens can include an accommodating lens, an optical axis passing through the accommodating lens a haptic portion positioned radially-outward from the accommodating lens, a cavity formed in the haptic portion and extending radially-outward with respect to the optical axis, and/or a visual marker positioned anterior to the cavity. The removable lens can be configured to removably couple with the base lens and have a lens portion and a tab extending radially-outward from the lens portion and configured to extend at least partially into the cavity when the removable lens is coupled to the base lens. In some embodiments, at least a portion of the tab is positioned posterior to and at least partially hidden from view from an anterior-posterior perspective when the removable lens is coupled to the base lens.

### CROSS-REFERENCE TO RELATED APPLICATION

This application claims priority to U.S. Provisional Patent Application No. 63/178,275, titled ACCOMMODATING INTRAOCULAR LENSES AND ASSOCIATED METHODS, filed Apr. 22, 2021, the disclosure of which is incorporated herein by reference in its entirety.

### TECHNICAL FIELD

The present technology relates to accommodating intraocular lenses (AIOLs) and methods of implanting and assembling the same.

### BACKGROUND

Cataracts can affect a large percentage of the worldwide adult population with clouding of the native crystalline lens and resulting loss of vision. Patients with cataracts can be treated by native lens removal and surgical implantation of a synthetic intraocular lens (IOL).

Worldwide, there are millions of IOL implantation procedures performed annually. In the U.S., there are 3.5 million cataract procedures performed, while worldwide there are over 20 million annual procedures performed.

Although IOL implantation procedures can be effective at restoring vision, conventional IOLs have several drawbacks. For example, many prior IOLs are not able to change focus as a natural lens would (known as accommodation). Other drawbacks of conventional IOLs include refractive errors that occur after implantation and require glasses for correcting distance vision, or in other cases the IOLs can be effective in providing good far vision, but patients need glasses for intermediate and near vision.

Several multi-focal IOLs have been developed to address these drawbacks, but they too can have drawbacks. For example, although multi-focal IOLs generally perform well for reading and distance vision, in at least some instances such multi-focal IOLs may cause significant glare, halos, reduced contrast sensitivity, and other visual artifacts.

AIOLs have been proposed to provide accommodative optical power in response to the distance at which a patient views an object. However, such AIOLs are generally still in development and have different drawbacks. For example, prior AIOLs can provide insufficient accommodation after implantation or produce suboptimal refractive correction of the eye. The amount of accommodation of the prior AIOLs can also decrease after implantation in at least some instances. The prior AIOLs can also be too large to be inserted through a small incision of the eye and may require the incision to be somewhat larger than would be ideal. Also, at least some of the prior AIOLs can be unstable when placed in the eye, which can lead to incorrect accommodation and other errors.

Improved implantable intraocular lenses that accommodate with the natural mechanisms of controlling focusing of the eye that overcome at least some of the above deficiencies would be desirable. Ideally, such improved AIOLs would provide increased amounts of accommodation when implanted, provide refractive stability, introduce few if any perceptible visual artifacts, and allow the optical power of the eye to change from far vision to near vision in response to the distance of the object viewed by the patient.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A illustrates an anterior-posterior elevation view of an embodiment of an AIOL.

FIG. 1B illustrates the accommodating structure of the AIOL of FIG. 1A in an exploded configuration.

FIG. 1C illustrates a cross-sectional view of the AIOL of FIG. 1A along cut-plane A-A of FIG. 1A.

FIG. 1D illustrates a cross-sectional view of the AIOL of FIG. 1A along cut-plane B-B of FIG. 1A.

FIG. 2 illustrates a plan view of the anterior face of an AIOL configured in accordance with an embodiment of the present technology.

FIG. 3A illustrates an exploded perspective view of an AIOL configured in accordance with an embodiment of the present technology.

FIGS. 3B and 3C illustrate perspective views of the AIOL of FIG. 3A when the fixed lens is received in receiving structures of the accommodating structure.

FIG. 3D illustrates a cross-sectional view of the AIOL of FIG. 3C taken along the optical axis of the AIOL of FIG. 3A.

FIG. 3E illustrates a top cross-sectional view of the AIOL of FIG. 3A with bumps on an inner surface of the accommodating portion and with the fixed lens in a first position.

FIG. 3F illustrates the AIOL of FIG. 3E with the fixed lens in a second position.

FIG. 3G illustrates a top cross-sectional view of the AIOL of FIG. 3A with steps on an inner surface of the accommodating portion and with the fixed lens in a first position.

FIG. 3H illustrates the AIOL of FIG. 3G with the fixed lens in a second position.

FIG. 4A illustrates an exploded perspective view of an AIOL configured in accordance with an embodiment of the present technology.

FIG. 4B illustrates a perspective view of the AIOL of FIG. 4A.

FIG. 5A illustrates an exploded view of an AIOL configured in accordance with an embodiment of the present technology.

FIG. 5B illustrates the AIOL of FIG. 5A wherein a fixed lens of the AIOL is coupled to a base lens of the AIOL.

FIG. 5C illustrates a plan view of the anterior face of the AIOL of FIG. 5B.

FIG. 5D illustrates a lateral cross-sectional view of the AIOL of FIG. 5B, taken along the cut-plane 5D-5D of FIG. 5C.

FIG. 6A illustrates an exploded perspective view of an AIOL configured in accordance with an embodiment of the present technology.

FIG. 6B illustrates a plan view of the anterior face of the AIOL of FIG. 6A.

FIG. 6C illustrates a perspective view of an AIOL configured in accordance with an embodiment of the present technology.

FIG. 6D illustrates a plan view of the anterior face of the AIOL of FIG. 6C.

### DETAILED DESCRIPTION

The present technology is directed to AIOLs and methods for making and using such devices. In many of the embodiments disclosed herein, the AIOLs include an accommodating lens portion (e.g., a base lens) and a fixed lens portion (e.g., a removable lens) configured to removably connect to the accommodating lens portion. The accommodating lens portion can include one or more visual indicators to aid a practitioner in connecting the fixed lens portion with the accommodating lens portion. In some embodiments, the visual indicator is a colored, tinted (e.g., opaque), or otherwise visually-modified structure that covers or hides a portion of the fixed lens (e.g., a portion of one or more tabs or other structure) when the fixed lens portion is fully connected to the accommodating lens portion. This visual confirmation of full connection between the two lens portions is expected to reduce the risk that the fixed lens portion is loose or otherwise insufficiently-connected to the accommodating lens portion during assembly, and is also expected to speed up the process of assembling the AIOL.

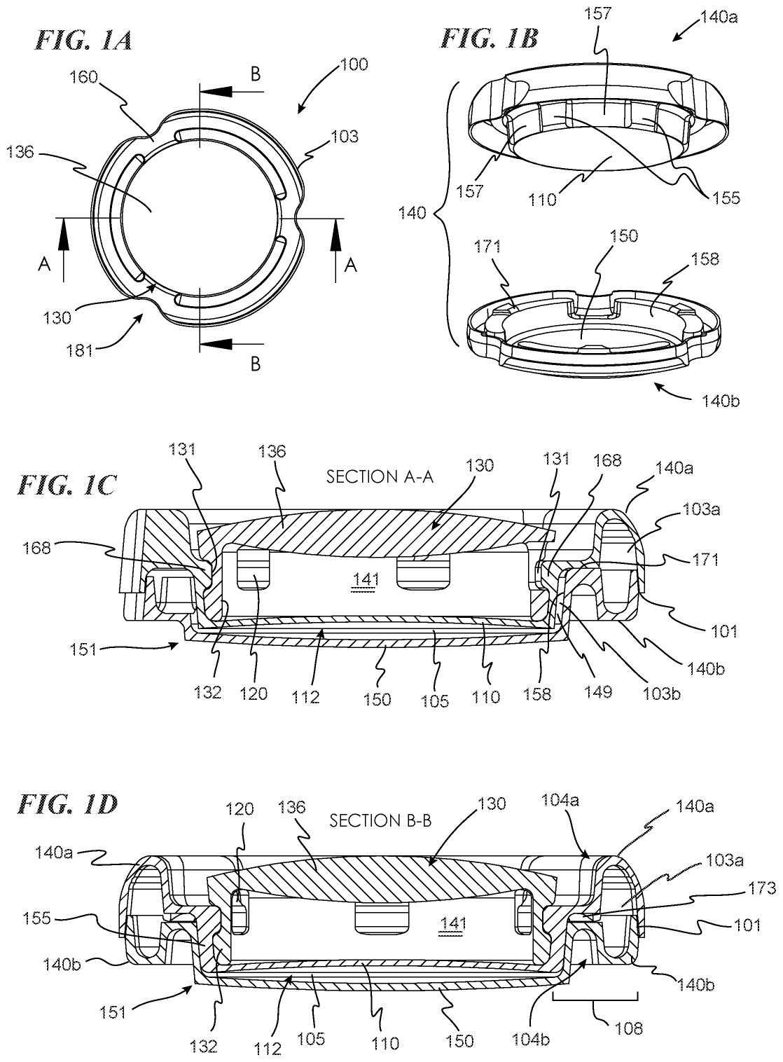

Specific details of various embodiments of the present technology are described below with reference to FIGS. 1A-6D. Although many of the embodiments are described below with respect to AIOLs and associated methods, other embodiments are within the scope of the present technology. Additionally, other embodiments of the present technology can have different configurations, components, and/or procedures than those described herein. For instance, AIOLs configured in accordance with the present technology may include additional elements and features beyond those described herein, or other embodiments may not include several of the elements and features shown and described herein.

For ease of reference, throughout this disclosure identical reference numbers are used to identify similar or analogous components or features, but the use of the same reference number does not imply that the parts should be construed to be identical. Indeed, in many examples described herein, the identically numbered parts are distinct in structure and/or function.

FIGS. 1A-ID illustrate an embodiment of an AIOL 100 including channels for fluid to flow from an outer fluid reservoir to an inner fluid chamber. Referring to FIGS. 1A and 1B together, the AIOL 100 includes an accommodating structure 140 (e.g., a base lens) having a first component 140a (e.g., an anterior lens component) and a second component 140b (e.g., a posterior lens component). In some embodiments, the first and second components 140a and 140b are assembled to form an outer fluid reservoir 103 (e.g., a haptic reservoir) (FIG. 1A), a mid-bellows channel 173 (FIG. 1D), and an inner fluid chamber 105 (e.g., an optical chamber) (FIGS. 1C-ID). The first component 140a of the accommodating structure 140 can have an inner portion with a first optical component 110, standoffs 155, and recesses 157 between the standoffs 155. The standoffs 155 project radially outward from the recesses 157. The second component 140b of the accommodating structure 140 can have an inner portion with a second optical component 150 and a wall 158. Referring to FIGS. 1C and 1D, which are cross-sectional views taken along lines A-A and B-B of FIG. 1A, respectively, the standoffs 155 contact the wall 158 (FIG. 1D) such that the recesses 157 (FIG. 1B) define channels for fluid to flow from the mid-bellows channel 173 to the fluid chamber 105.

As illustrated in FIG. 1D, the standoffs 155 project radially outward to engage the wall 158. The standoffs 155 of the AIOL 100 accordingly do not extend into the optical region of the AIOL, which increases the field of view of the AIOL 100.

Referring again to FIGS. 1B and 1C, one or both of the first and second components 140a, 140b of the accommodating structure 140 can include axial projections, standoffs, spacers, protrusions, or other features configured to space a portion of the first component 140a from the second component 140b in a direction parallel to an optical axis of the AIOL 100. For example, the second component 140b can include one or more projections 171 extending from an anterior edge of the wall 158 toward the first component 140a. Space between the projections 171 can form the mid-bellows channels 173 described below. In some embodiments, one or both of the first and second components 140a, 140b include indentations that define, at least in part, the mid-bellows channels 173. Such indentations can be included in addition to or instead of the projections 171. For example, the anterior edge of the wall 158 can include one or more indentations and/or channels.

In some embodiments, the AIOL 100 includes flow-through features 181 that enhance the rate and ease with which Ophthalmic Viscosurgical Devices (OVDs) used during the implantation of AIOLs can be removed from the natural lens capsule. The embodiment of the AIOL 100 illustrated in FIGS. 1A-ID comprises three outer flow-through features 181. The outer flow-through features 181 can be detents, such as a recess, distributed circumferentially along the perimeter of the outer fluid reservoir 103. The flow-through features 181 can create passages between the outer perimeter of the AIOL 100 and an inner surface of an eye capsule in which the AIOL 100 is implanted to allow fluid flow around an outer perimeter of the AIOL 100. In the illustrated embodiment, the flow-through features 181 are formed in regions of the first and second components 140a and 140b. Although three outer flow-through features 181 are illustrated, other embodiments may comprise fewer or more than illustrated. The outer flow-through features 181 may additionally provide rotational constraint to maintain the rotational orientation of the accommodating structure 140 with respect to a patient's eye capsule when implanted.

The embodiment of the AIOL 100 additionally comprises a fixed lens assembly 130. The fixed lens assembly 130 illustrated in FIGS. 1C-D includes an optical portion 136, a skirt 132 extending from the optical portion 136, and passages 120. The optical portion 136 can have a fixed power which may comprise an asymmetrically powered lens (e.g., a toric lens) or other lens, and the passages 120 are holes, slots, orifices, etc., that pass through the skirt 132 and extend into a perimeter region but not the optical portion 136.

Referring to FIG. 1C, the fixed lens assembly 130 can have an engagement feature 131, such as an annular groove, that extends around the skirt 132, and the first component 140a of the accommodating structure 140 can have a thickened region 168, such as an annular protrusion (e.g., a ledge) that extends radially inwardly. The fixed lens assembly 130 can be releasably attached to the accommodating structure 140 by engaging the continuous thickened region 168 of the first component 140a with the engagement feature 131 of the fixed lens 130. In other embodiments (not shown), the thickened region 168 and the engagement feature 131 may be discontinuous features (e.g., segmented or other recesses or protrusions that extend around less than the full circumference of the fixed lens assembly 130 and the accommodating structure 140). Such a discontinuous thickened region 168 and engagement feature 131 can facilitate maintenance of a particular radial alignment between the fixed lens assembly 130 and the accommodating structure 140, such as when the fixed lens 130 comprises a toric lens or other asymmetrical lens. Alternatively, the groove may be in the fixed lens 130 and the protrusion on the accommodating structure 140.

The AIOL 100 can have a fluid accommodating lens 112 defined by a fluid chamber 105 (FIGS. 1C and 1D) bounded between a first optical component 110 and a second optical component 150. The fluid chamber 105 is in fluid communication with the outer reservoir 103 via discrete fluid channels 149 between standoffs 155 when the first and second components 140a and 140b are assembled. The first and second optical components 110 and 150 may be planar members (e.g., optical membranes) of the first and second components 140a and 140b, respectively. The first and second optical components 110 and 150, for example, can be integrally formed as optical membranes with the other portions of the first and second components 140a and 140b. In alternate embodiments, either or both of the membranes of the first and second optical components 110 and 150 may be a lens (i.e., have an optical power).

The AIOL 100 can further include a square-shaped (e.g., stepped) annular region 151 that inhibits cell migration from the periphery of the patient's capsule to the optical part of AIOL 100 (shown in FIGS. 1C-D at the posterior most region of the lens). Inhibiting cell migration from the periphery of the patient's capsule to the optical part of the AIOL 100 can reduce the risk of post-surgery opacification of the optical system.

The peripheral portions of the first component 140a and the second component 140b define the outer fluid reservoir 103, and the inner portions of the first and second components 140a and 140b define the accommodating structural element 140. The first and second components 140a and 140b can be bonded together at a seam 101. Means of bonding are described in detail in PCT Pub. No. WO2018/119408, appended to the end of the present disclosure. The first and second components 140a and 140b can also be bonded at other areas, such as at the standoffs 155. The standoffs 155 are separated by spaces that define fluid channels between the outer fluid reservoir 103 and the inner fluid chamber 105. The outer fluid reservoir 103 can be a bellows 108 having an outer bellows region 103a and an inner bellows region 103b, and the inner bellows region 103b can be defined by the channels between the standoffs 155.

In some embodiments, the volume of the inner bellows region 103b is less than the outer bellows region 103a. By reducing the volume of the inner bellows region 103b, additional space surrounding the optical region of the AIOL allows the optical aperture of the fixed lens 130 to be larger compared to embodiments with larger inner bellows regions. Additionally, the passages 120 of the fixed lens 130, which allow aqueous fluid to freely flow in and out of the chamber 141, are configured to pass through the outer skirt 132 and, in some embodiments, not the top optical portion 136. This is expected to reduce unwanted scattered light from internal reflections which may pass through the optical system and reach the retina.

The first component 140a may also comprise one or more thickened regions 160 for use, for instance, in filling the AIOL with an optical fluid. The thickened region 160 allows for a longer path for a needle used to fill the accommodating structure with optical fluid while a second needle in a different region is used to remove the gases the fluid is replacing. As illustrated, the fluid fill thickened region 160 is located adjacent one or more of the outer fluid flow-throughs 181. In some embodiments, the optical fluid may be comprised of a high refractive index poly vinyl alcohol.

Referring to FIG. 1D, the outer fluid reservoir 103 of the AIOL 100 can comprise (a) a first bellows structure 103a with an anterior portion 104a and a posterior portion 104b, (b) a second bellows structure 103b radially inward of the first bellows structure 103a, and/or (c) the mid-bellows channel 173 defining a horizontal passageway between the first and second bellows structures 103a and 103b. During operation as the capsule contracts, a mid-portion of the first bellows structure 103a can be constrained by the mid-bellows channel 173 while the anterior and posterior portions 104a and 104b of the first bellows structure 103a move radially inward with respect to the mid-bellows channel 173. The anterior and posterior portions 104a and 104b of the first bellows structure 103a can accordingly flex radially inward in response to the same amount of movement of the native capsule. This can cause more fluid to flow from the outer fluid reservoir 103 to the inner fluid chamber 105 and thereby provides more accommodation because anterior-posterior collapse of the outer fluid reservoir 103 is less efficient than radial compression of the outer fluid reservoir 103. Embodiments such as, but not limited to, any of those illustrated herein may be constructed from parts in which some or all of the portions not in the optical path have been dyed or treated to reduce light throughout to limit the ability of stray light entering portions outside the optical path from scattering into the optical path.

The fixed lens described in any of the embodiments described herein may be of spherical, aspheric, toric, or any other known lens configuration. Alternatively, or in combination, the fixed solid lens may be plano-convex, convex-concave, or convex-convex. The fixed lens may be configured to have positive or have negative fixed power.

The fluid lenses described herein may be configured such as to have one or more accommodating surfaces (e.g., two accommodating surfaces).

In some embodiments, instead of membranes without a power, the accommodating structure can include one or more deformable lenses that deflect based upon fluid pressure within the inner fluid chamber. The deformable lenses can each or both have a fixed power that can be positive or negative.

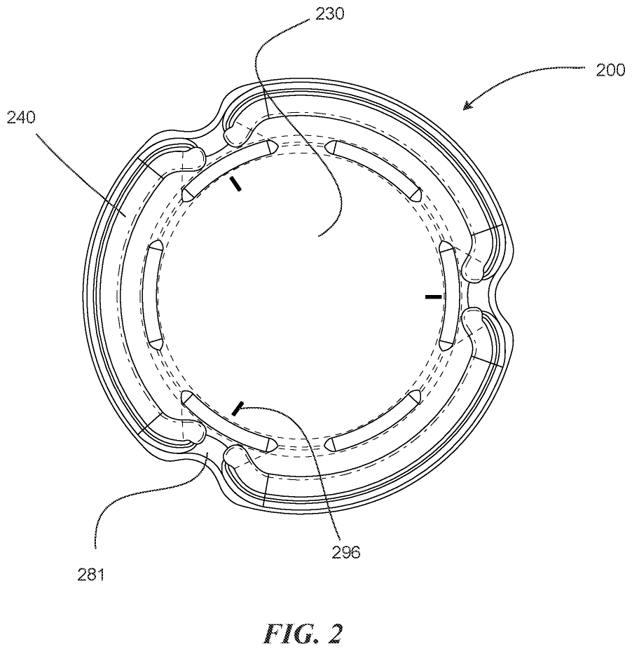

FIG. 2 illustrates an AIOL 200 configured in accordance with another embodiment of the present technology. The AIOL 200 has many or all of the same features of the AIOL 100 described above. In some embodiments, for example, the accommodating structure 240 of the AIOL 200 is identical to or similar to the accommodating structure 140 of AIOL 100. The accommodating structure 240 of the AIOL 200 can have markings 296 positioned on one or both of the first and second optical components of the accommodating structure 240. The markings 296 can be viewable from outside of the patient's eye and or through the fixed lens 230 of the AIOL 200. The markings 296 allow a surgeon or other medical personnel to locate the flow-through features 281 (e.g., indentations) or other specific features of the AIOL 200. For example, the AIOL 200 can include a marking 296 aligned with each of the flow-through features 281. Preferably, the markings 296 are positioned at or near a perimeter of the optical portion of the AIOL to avoid optical distortions for the patient. Accurately and reliably locating the flow-through features 281 can allow medical personnel to position cannulas or other flushing devices in orientations to efficiently flush OVD or other materials from the eye capsule posterior to the AIOL 200. For example, knowing the position of the flow-through features 281 can allow medical personnel to direct flushing fluid toward the flow-through features 281 when flushing the portion of the lens capsule posterior of the AIOL 200.

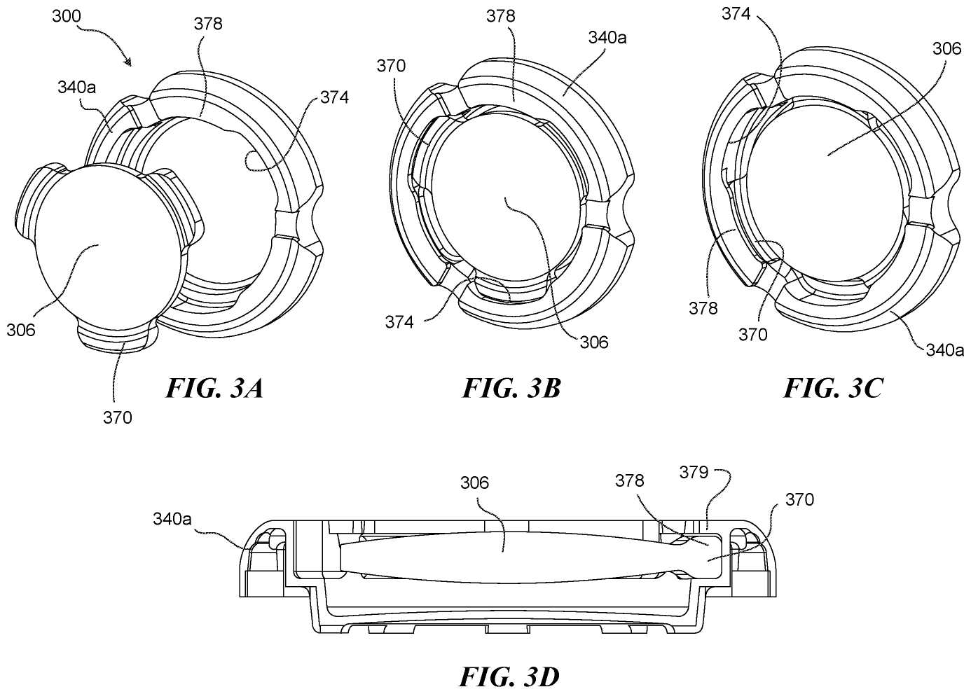

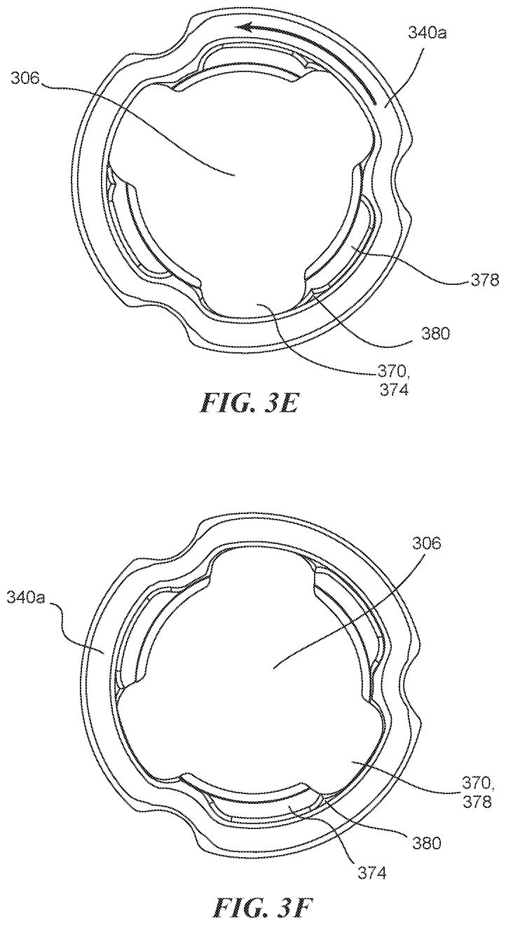

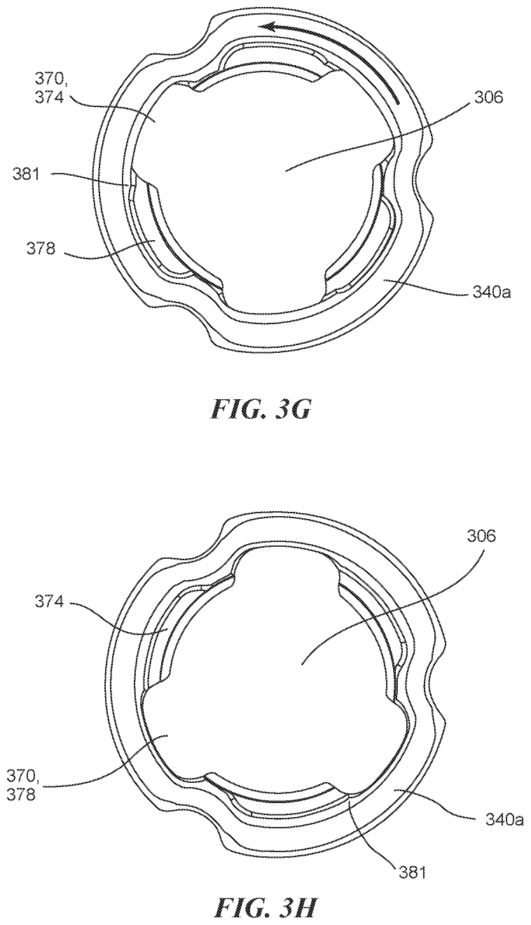

FIGS. 3A-3D illustrate an AIOL 300 configured in accordance with another embodiment of the present technology. The AIOL 300 can include a number of features similar to or the same as the features of the AIOL 100 described above with reference to FIGS. 1A-ID. Accordingly, like numbered components can be similar to or the same as each other (e.g., first component 140a v. first component 340a). The AIOL 300 can include a fixed lens 306 having one or more mating structures 370) extending from an optical portion of the fixed lens 306. The mating structures 370 can be, for example, fins, tabs, protrusions, flanges, extensions, or other structures extending from the optical portion of the fixed lens 306 in a direction perpendicular to or generally perpendicular to an optical axis of the fixed lens 306. In the illustrated embodiments, the fixed lens 306 includes three mating structures 370 evenly distributed in a circumferential pattern around the perimeter of the optical portion of the fixed lens 306. In some embodiments, more or fewer mating structures are used, and patterns other than even circumferential distribution may be used. The first component 340a (e.g., an anterior component) of the accommodating structure (e.g., the base lens) of the AIOL 300 can include one or more receiving structures 374 configured to receive and releasably mate with the mating structures 370 of the fixed lens 306. While the second component of the accommodating structure is not illustrated, it can be identical or similar to the second component 140b described above. One or more of the receiving structures 374 can be, for example, a slot or other indentation configured to receive corresponding mating structures 370 (e.g., in a direction parallel to the optical axis of the AIOL 300), as illustrated in FIG. 3B. The first component 340a can include a retaining structure 378 (e.g., one retaining structure 378 for each receiving structure 374) configured to secure the fixed lens 306 to the first component 340a when the fixed lens 306 is fully coupled with the first component 340a. The retaining structures 378 can be, for example, pockets, depressions, indentations, cavities, or other structures configured to retain at least a portion of the mating structures 370 of the fixed lens 306. These structures for mating with the fixed lens may singular or plural structures. In the illustrated embodiment, the retaining structures 378 are circumferentially-extending cavities extending from the receiving structures 374. After the mating structures 370) are received in/through the receiving structures 374 (e.g., slots), the fixed lens 306 can be rotated (e.g., in a counterclockwise direction) such that the mating structures 370 are at least partially received and retained in the retaining structures 378 (FIGS. 3C-3D).

The retaining structures 378 can be configured to inhibit or prevent inadvertent decoupling of the fixed lens 306 from the first component 340a. For example, the retaining structures 378 can include anterior walls 379 (FIG. 3D) positioned to inhibit or otherwise interfere with movement of the mating structures 370 when the mating structures 370 are positioned at least partially within the retaining structures 378.

In some embodiments, the mating structures 370) are sized/shaped to deflect a portion of the retaining structures 378 and/or increase friction between the mating structures and the retaining structures 378 when the mating structures are positioned at least partially within the retaining structures 378. For example, a portion of the mating structures 370 can be larger in one or more dimensions (e.g., parallel to and/or perpendicular to the optical axis of the AIOL 300) than the retaining structures 378 such that one or more of the retaining structures 378 are deflected and/or deformed when a mating structure 370 is received therein.

FIGS. 3E-3H illustrate additional features that may be used in combination with the above-described AIOL 300. For example, referring to FIGS. 3E-3F, the first component 340a can include a bump 380, protrusion, or other structure configured to interfere with rotation of the fixed lens 306 when the mating structures 370 are positioned at least partially within the retaining structures 378. The bumps 380 can be positioned on a radially-inward wall of the first component 340a between the receiving structure 374 and the retaining structure 378. The bumps 380 can be configured to deflect outward as the mating structures 370 move from the receiving structures 374 to the retaining structures 378 (e.g., as the fixed lens 306 is rotated with respect to the first component 340a, as indicated by the arcuate arrow in FIG. 3E). Once the mating structures 370 pass the bumps 380, the bumps 380 can deflect radially inward to interfere with rotation of the mating structures 370 back to the receiving structures 374.

FIGS. 3G and 3H illustrate a feature that can be used instead of, or in addition to the above-described bump 380. Specifically, the receiving structure 374 can have a radius (e.g., as measured perpendicular to the optical axis of the AIOL 300) that is smaller than a radius of the retaining structure 378. The radius of the receiving structure 374 can be smaller than the radius of the mating structures 370 of the fixed lens 306. In some embodiments, when the fixed lens 306, or the mating structures 370 thereof, are received in the receiving structures 374, the mating structures 370) can deflect the receiving structures 374 in a radially-outward direction. When the mating structures 370) are moved into the retaining structures 378 (see, e.g., the arcuate arrow in FIG. 3G), the receiving structures 374 can return to their undeflected positions. Steps 381 between the radius of the receiving structures 374 and the radius of the retaining structures 378 are positioned to inhibit or prevent inadvertent movement of the mating structures 370) from the retaining structures 378 to the receiving structures 374.

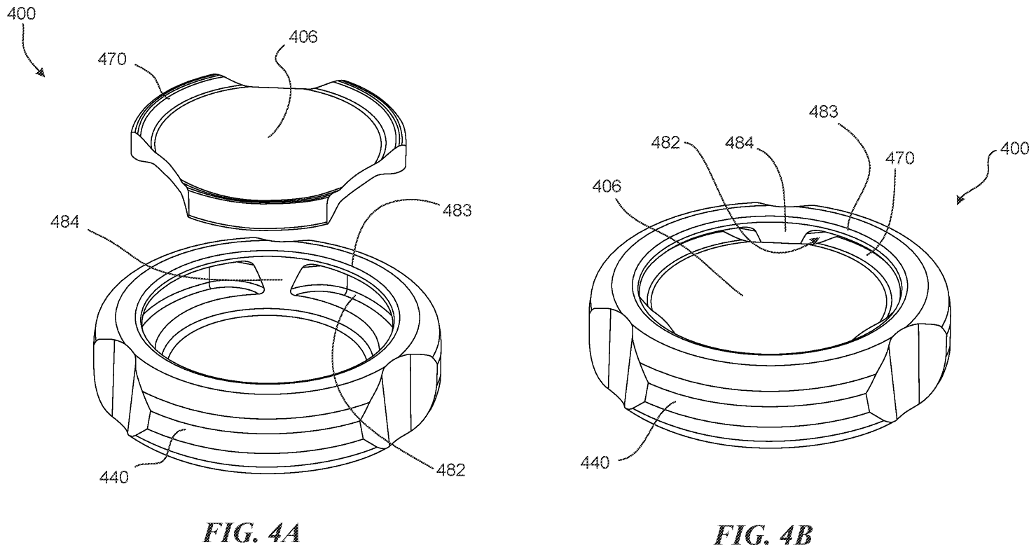

FIGS. 4A-4B illustrate an AIOL 400 configured in accordance with another embodiment of the present technology. The AIOL 400 can include a number of features similar to or the same as the features of the above-described AIOLs (e.g., AIOLs 100 and 300). Thus, like numbered components can be similar to or the same as each other (e.g., fixed lens 306 v. fixed lens 406). The accommodating structure 440 of the AIOL 400 can include a plurality of cavities 482 or other cavities configured to releasably receive mating structures 470 (e.g., fins, protrusions, flanges, or other mating structures) of the fixed lens 406. The cavities 482 can include anterior walls 483. The anterior walls 483 can be configured to bend, deflect, and/or otherwise deform to allow the mating structures 470 of the fixed lens 406 to pass at least partially into and out of the cavities 482. The accommodating structure 440 can include interior walls 484 separating the cavities 482. The interior walls 484 can inhibit or prevent free rotation of the fixed lens 406 when the fixed lens 406 is mated with the accommodating structure 440 (as shown in FIG. 4B).

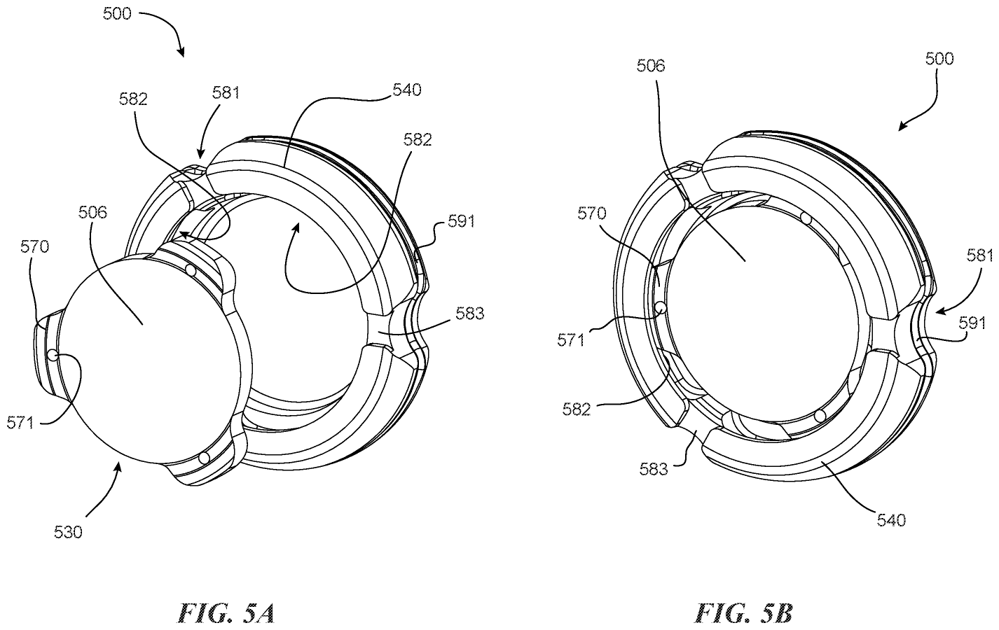

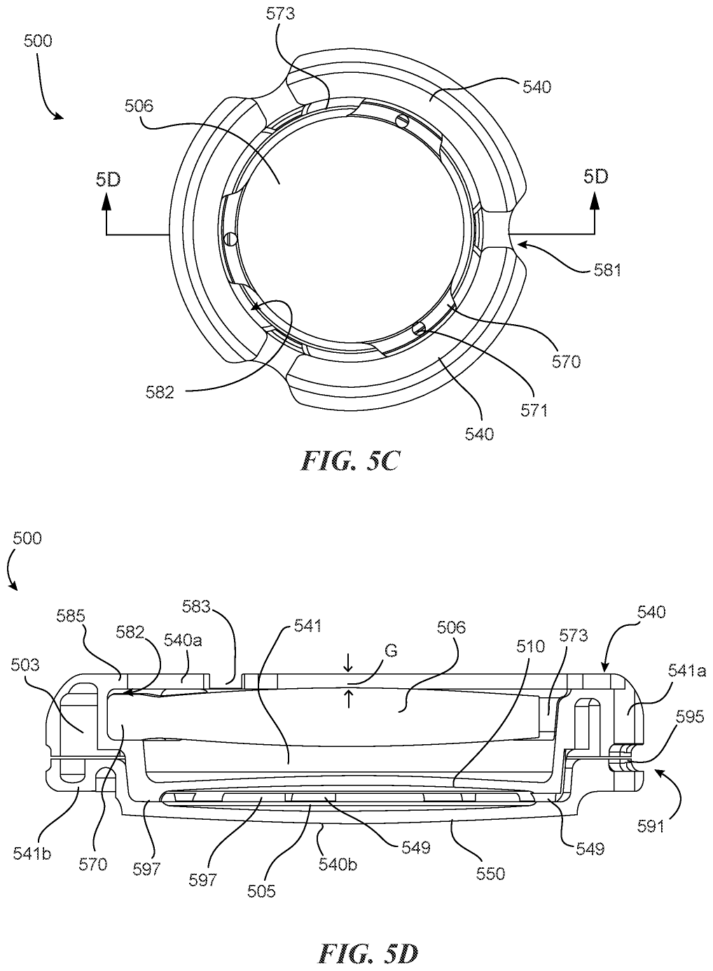

FIGS. 5A-5D illustrate an AIOL 500 configured in accordance with another embodiment of the present technology. The AIOL 500 can include a number features similar to or the same as the features of AIOLs 100, 300, and 400 described above. Accordingly, like numbered components can be similar to or the same as each other (e.g., cavities 482 v. cavities 582, fixed lens 406 v. fixed lens 506, etc.). Referring to FIGS. 5A and 5B, the fixed lens assembly 530 can include a lens portion 506 (e.g., a fixed lens) and one or more tabs 570) extending radially outward from the lens portion 506. One or more holes 571 can extend at least partially through the one or more tabs 570. The holes 571 (e.g., or indentation) can be engaged by a surgical tool to manipulate the fixed lens assembly 530 during implantation and/or removal of the fixed lens assembly 530. In some embodiments, the holes 571 are arranged in pairs. In some embodiments, the holes 571 are distributed in a circumferential pattern. In some embodiments, one or more of the holes 571 has a different size (e.g., width or diameter) and/or shape than other holes 571. For example, each of the holes 571 may have a different size than each of the other holes 571. Using holes of varying size can allow for further visual confirmation of the rotational alignment of the fixed lens assembly 530 (e.g., about the optical axis of the AIOL 500). Confirming the alignment/orientation of the fixed lens assembly 530 can reduce the risk that a toric lens or other non-annularly-symmetric fixed lens 506 is improperly oriented with respect to the base lens 540 and/or native eye capsule into which the AIOL 500 is implanted. In some embodiments, the fixed lens assembly 530 can include additional visual markers (e.g., similar to or the same as the markings 896 described above with respect to FIG. 8) to indicate an orientation of the fixed lens assembly 530 with respect to the base lens 540 and/or the native eye capsule.

The AIOL 500 further comprises a base lens 540) (e.g., accommodating structure). As best seen in FIG. 5A, the base lens 540 can include one or more cavities 582 or other mating structures configured to releasably couple with the one or more tabs 570 of the fixed lens assembly 530. The cavities 582 can open toward an optical axis of the base lens 540. In some embodiments, there are three cavities 582 to receive three tabs 570 of the fixed lens assembly 530. The cavities 582 can be positioned in portions of the base lens 540 and separated from each other by the flow-through features 581 and/or walls adjacent the indentations 583 of the base lens 540. The indentations 583 can be aligned with flow-through features 581 of the base lens 540 having similar or identical features to the flow-through features 181 described above. The indentations 583 and/or flow-through features 581 can facilitate flow of fluid around the outer perimeter of the base lens 540 when the base lens 540 is implanted in the eye capsule of a patient. When the AIOL is assembled, the tabs 570, fixed lens 506, and/or the cavities 582 can be coplanar with each other.

The base lens 540) can include an outer channel 591 on a radially-outer surface of the base lens 540 (e.g., on the anterior base lens component 540a and/or posterior base lens component 540b, as illustrated in FIG. 5D). The outer channel 591 can extend around the entire perimeter of the base lens 540. In some embodiments, the outer channel 591 extends along a seam 595 (FIG. 5D) between the anterior and posterior base lens components 540a, 540b. The outer channel 591 can facilitate increased fluid flow and/or flow of OVD around the outside of the base lens 540 when the base lens is implanted in the eye capsule of a patient. In some embodiments, the outer channel 591 can reduce the likelihood that the base lens 540 adheres to the inner wall of the eye capsule when implanted.

FIG. 5C is a plan view of the anterior face of the AIOL 500, and FIG. 5D illustrates a lateral cross-sectional view of the AIOL 500 taken along the cut-plane 5D-5D of FIG. 5C. As best seen in FIG. 5D, the cavities 582 can be at least partially defined by anterior flanges 585. The flanges 585 can overlap the tabs 570 in a direction parallel to the optical axis of the of the base lens 540 when the tabs 570 are positioned within the cavities 582. The flanges 585 can inhibit or prevent inadvertent (e.g., under the forces of the native eye capsule) decoupling of the fixed lens assembly 530 from the base lens 540.

In some embodiments, the circumferential spaces between the tabs 570 can at least partially define passages 573 between the fixed lens 506 and the base lens 540 when the fixed lens assembly 530 is coupled to the base lens 540. The passages 573 can permit fluid (e.g., aqueous humor) to pass into and out from a chamber 541 between the fixed lens 506 and the optical portion 510 of the anterior base lens component 540a.

As illustrated in FIG. 5D, the haptic reservoir 503 of the base lens 540 can be defined at least in part by the haptic portions 541a, 541b of the anterior and posterior base lens components 540a, 540b, respectively. The haptic reservoir 503 can be in fluid communication with a fluid chamber 505 between the optical portion 510 of the anterior base lens component 540a and the optical portion 550 of the posterior base lens component 540b. This fluid communication can be facilitated by one or more fluid flow paths 549. These fluid flow paths 549 can be defined by gaps between protrusions 597 (e.g., standoffs) along the perimeter of the optical portion 510 of the anterior base lens component 540a. These protrusions 597 can form passages 549 therebetween when the anterior base lens component 540a is mated with the posterior base lens component 540b. In some embodiments, the posterior base lens component 540b includes protrusions or standoffs to form the fluid flow paths 549. These protrusions can extend from a perimeter of the optical portion 550 of the posterior base lens component 540b and can be used in addition to or instead of the protrusions 597 described above.

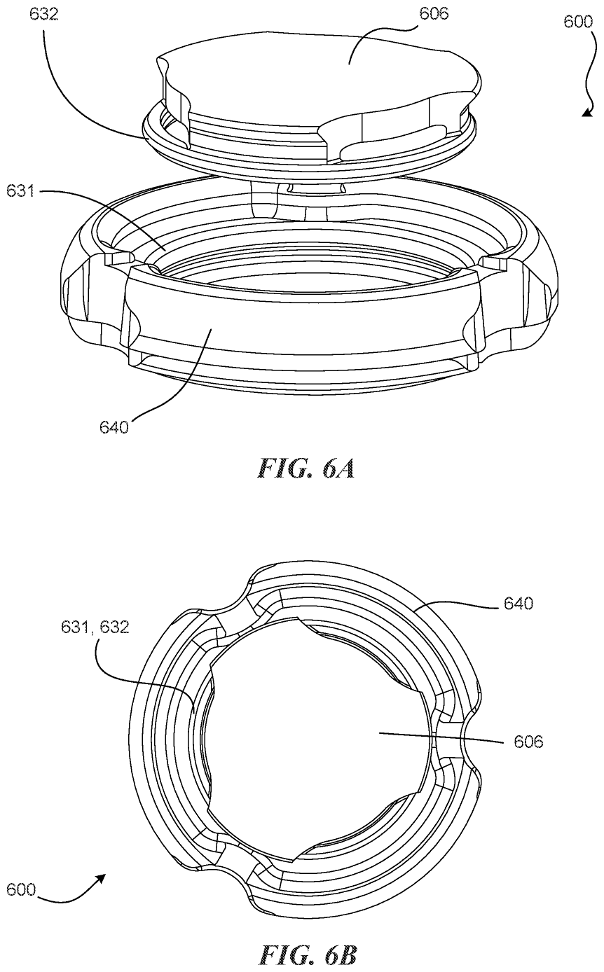

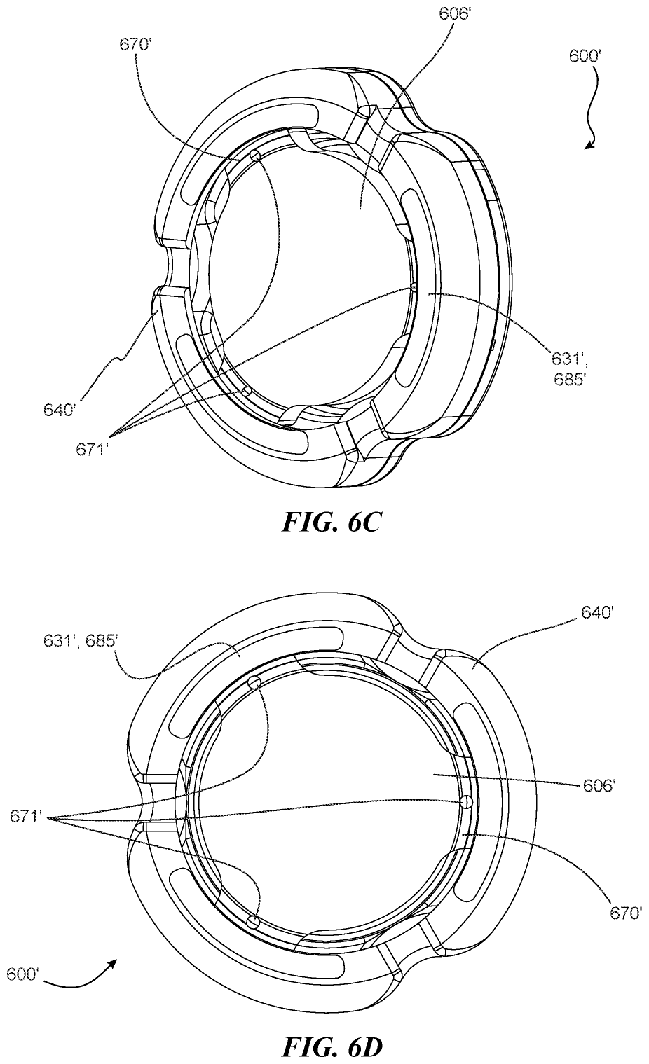

FIGS. 6A-6B illustrate an AIOL 600 configured in accordance with another embodiment of the present technology. The AIOL 600 can include a number of features similar to or the same as the features of the AIOL 100 described above. Accordingly, like numbered components can be similar to or the same as each other (e.g., accommodating structure 140 v. accommodating structure 640). The fixed lens 606 of the AIOL 600 can include a skirt 632 sized and shaped to engage with an engagement feature 631 of the accommodating structure 640 (e.g., the base lens) in a manner similar to or the same as that described above with respect to the engagement feature 131 and skirt 132 of FIG. 1C-1D. In the embodiment shown in FIGS. 6A and 6B, all or a portion of the skirt 632 (e.g., a visual marker) can be colored in a first color. All or a portion of the engagement feature 631 (e.g., a visual marker) can be colored in a second color such that, when the fixed lens 606 is mated with the accommodating structure 640, a third color is observed from a position anterior of the AIOL 600 (FIG. 6B) (e.g., through the engagement feature 631). The third color is created from the overlap in the anterior-posterior direction of the skirt 632 and the engagement feature 631. For example, the first color can be blue and the second color can be yellow such that the third color is green. Other first and second color combinations (e.g., red-yellow, red-blue, etc.) can also be used. In some embodiments, the entire circumference of the skirt 632 is colored with the first color so that full annular engagement between the fixed lens 606 and the accommodating structure 640 can be visually confirmed.

FIGS. 6C-6D illustrate an AIOL 600′ configured in accordance with another embodiment of the present technology. The AIOL 600′ can include a number of features similar to or the same as the features of the AIOLs 500 and 600 described above. Accordingly, like numbered components can be similar to or the same as each other (e.g., fixed lens 506 v. fixed lens 606′). The accommodating structure 640′ (e.g., part of a base lens of the AIOL 640′) can include one or more visual indicator portions 631′ (e.g., visual markers). The visual indicator portions 631′ can be, for example, colored, tinted (e.g., translucent or opaque), or otherwise visually-modified portions of the accommodating structure 640′. For example, the visual indicator portions 631′ can have a higher opacity, lower transparency, or otherwise different visual attributes than the surrounding portions of the accommodating structure 640′. In some embodiments, the visual indicator portions 631′ are portions of the accommodating structure 640′ (e.g., portions of the anterior flanges 685′) that are of a different color and/or opacity from the portions of the accommodating structure 640′ adjacent and/or surrounding the visual indicator portions 631′. The visual indicator portions 631′ can be positioned radially-inward from the bellows portions of the accommodating structure 640′.

In some embodiments, the visual indicator portions 631′ are formed by impregnating and/or coating the accommodating structure 640′ with a dye or other colorant. In some embodiments, one or more chemicals or other catalysts (e.g., heat) are used to change the opacity and/or color of one or more portions of the accommodating structure 640′ to form the visual indicator portions 631′. In some embodiments, the visual indicator portions 631′ are formed by applying (e.g., via adhering, co-molding, overmolding, etc.) separate structures to the accommodating structure 640′. The separate structures can be structures that are a different color and/or higher opacity than the accommodating structure 640′.

In some embodiments, the visual indicator portions 631′ are formed by partially curing darkened and/or colored silicone or other material in a mold. The partially-cured structures can be inserted into a mold used to mold the accommodating structure 640′. The partially-cured structure can be co-molded with the accommodating structure 640′ during manufacture.

As illustrated in FIG. 6D, the visual indicator portions 631′ can overlap and/or hide portions of the tabs 670′ of the fixed lens 606′ (e.g., the removable lens) when the fixed lens 606′ is fully coupled to the accommodating structure 640′. The tabs 670′ can be coplanar with the lens portion of the fixed lens 606′. A physician can confirm that the fixed lens 606′ is fully coupled to the accommodating structure 640′ when, from an anterior perspective, e.g., at least a portion of each of the tabs 670′ are at least partially hidden or covered by the visual indicator portions 631′ of the accommodating structure 640′. This can provide visual feedback to the physician regarding the state (e.g., coupled/decoupled) of the fixed lens 606′ during implantation, and reduce the risk that the fixed lens 606′ is not fully implanted.

In some embodiments, one or more of the visual indicator portions 631′ comprises a portion of the anterior sides of the accommodating structure 640′ having a different surface roughness or other optical characteristic from the portions of the accommodating structure 640′ surrounding the visual indicator portions 631′. For example, laser treatment, plasma etching, chemical etching, and/or mold features (e.g., bumps/roughness built into an anterior surface of the mold used to form the anterior accommodating structure 640′) can be used to manufacture the visual indicator portions 631′. In some embodiments, a different pattern, a hazy portion, and/or another unique visual/optical characteristic can be used to comprise the visual indicator portions 631.

The multipart AIOL devices described herein may be implanted by preparing the eye and removing the native lens from the capsule in any appropriate manner. The fluid-filled structure may then be placed in the capsule of the eye. The patient may then be evaluated for a base optical power and/or astigmatic correction, and a fixed lens is selected to provide the desired based power or astigmatic correction for the fluid-filled structure in the implanted state in the capsule of the eye. The specific fixed lens to provide the post-implant base power or astigmatic correction is then inserted into the previously implanted fluid-filled structure of the AIOL. The chosen fixed lens may then be coupled to the fluid-filled structure within the eye capsule. This is possible in the AIOLs of the present technology because the fixed lenses are attached to the anterior first component of the AIOLs. As described above, one or more of the fluid-filled accommodating structure or fixed lens may each be flexible such that they may be reconfigured (e.g., folded) to a reduced-profile delivery configuration for delivery into the lens capsule. In some instances, it may be required to make a further correction to the fixed portion after the time of the surgery. Such instance may occur anywhere from days to years after the surgery. At such times, the patient may return to the physician and the fixed lens may be replaced with a new fixed lens having a different optical power or other prescription. In such instances, the new prescription may be characterized prior to or after removal of the original fixed lens. In some instances, the new fixed lens may be fabricated and implanted at the time of the examination, in others the patient may return for implantation of the fixed lens sometime after the examination.

Several embodiments of the present technology are directed to a kit having an accommodating structure and a first fixed lens that has no optical base power. The kit can further include one or more second fixed lenses having various based powers or other optical properties. In practice, the accommodating structure can be implanted into the native eye capsule, and then the first fixed lens can be coupled to the accommodating structure. The optical properties of the implanted accommodating structure can then be assessed in situ with the first fixed lens in place to determine the desired optical properties of the fixed lens. If the optical properties of the assembled accommodating structure and first fixed lens without a base power are appropriate, then the system can remain implanted without additional changes. However, if a different base power or some other optical property is desired (e.g., toric or other asymmetrical optics), then the first fixed lens without a base power can be replaced with a second fixed lens having the desired optical properties based on the optical properties of the implanted accommodating portion with a fixed lens attached.

In some embodiments, the fixed portion of the AIOL may be fabricated from materials different from the accommodating portion. Such materials include hydrophilic or hydrophobic methacrylate or silicones and any other materials traditionally used in non-accommodating IOLs. The fixed lens may be fabricated from materials harder than those used for the accommodating portion. One or both of the accommodating portion/lens and the fixed portion/lens may be machined, cast molded (e.g., reactive cast molded), injected molded, and/or formed by other processes or combinations of processes. Any or all of the structures described herein may be constructed from a transparent or translucent material. For example, the above-described accommodating structures and fixed lenses can be constructed from transparent materials, even if they are illustrated as opaque in the associated figures.

Any of the features of the intraocular lens systems described herein may be combined with any of the features of the other intraocular lenses described herein and vice versa. Additionally, several specific examples of embodiments in accordance with the present technology are set forth below in the following examples.

### EXAMPLES

Several aspects of the present technology are set forth in the following examples.
1. An accommodating intraocular lens (AIOL) comprising:
a base lens having—
   an accommodating lens;
   an optical axis passing through the accommodating lens;
   a haptic portion positioned radially-outward from the accommodating lens with respect to the optical axis;
   a cavity formed in the haptic portion and extending radially-outward with respect to the optical axis; and
   a visual marker positioned anterior to the cavity; and
a removable lens configured to removably couple with the base lens, the removable lens having
   a lens portion; and
   a tab extending radially-outward from the lens portion and configured to extend at least partially into the cavity when the removable lens is coupled to the base lens, wherein at least a portion of the tab is positioned posterior to the visual marker and at least partially hidden from view from an anterior-posterior perspective when the removable lens is coupled to the base lens.
2. The AIOL of example 1 wherein the visual marker is a different color from at least a portion of base lens surrounding the visual marker.
3. The AIOL of example 1 wherein the visual marker is opaque.
4. The AIOL of example 1 wherein the visual marker is translucent
5. The AIOL of example 1 wherein the visual marker is less transparent than at least a portion of the base lens surrounding the visual marker.
6. The AIOL of example 1 wherein the visual marker comprises a darkened and/or colored silicone structure.
7. The AIOL of example 6 wherein the visual marker is formed in a mold separate from the base lens.
8. The AIOL of example 6 wherein the visual marker is partially cured and then co-molded with the base lens during manufacture.
9. The AIOL of example 1 wherein the visual marker is adhered to the base lens during manufacture.
10. The AIOL of example 1 wherein the visual marker is overmolded to the base lens during manufacture.
11. The AIOL of example 1 wherein the tab is coplanar with the lens portion of the removable lens.
12. A method of assembling an accommodating intraocular lens (AIOL), the method comprising:
providing a base lens, the base lens having
   an accommodating lens;
   an optical axis passing through the accommodating lens;
   a haptic portion positioned radially-outward from the accommodating lens with respect to the optical axis;
   a cavity formed in the haptic portion and extending radially-outward with respect to the optical axis; and
   a visual marker positioned anterior to the cavity
coupling a removable lens to the base lens by inserting a tab of the removable lens into the cavity such that the tab is positioned posterior of the visual marker and overlaps the marker in a direction parallel to the optical axis; and
aligning a lens portion of the removable lens with the optical axis.
13. The method of example 12 wherein the removable lens is coupled to the base lens after the base lens is implanted in an eye of a patient.
14. The method of example 12 wherein the visual marker hides a portion of the tab when observed from a position anterior to the visual marker.
15. The method of example 12, further comprising overmolding the visual marker to the base lens.
16. The method of example 12 wherein the visual marker is overmolded to the base lens.
17. The method of example 12 wherein the visual marker is less transparent than at least a portion of the base lens surrounding the visual marker.
18. The method of example 12 wherein the visual marker is opaque.
19. The method of example 12, further comprising adhering the visual marker to the base lens.
20. The method of example 12, further comprising co-molding the visual marker with the base lens.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the AIOLs described herein may be combined with any of the features of the other AIOLs described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with AIOLs have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An accommodating intraocular lens (AIOL) comprising:
a base lens having—
an accommodating lens;
an optical axis passing through the accommodating lens;
a haptic portion positioned radially-outward from the accommodating lens with respect to the optical axis;
a cavity formed in the haptic portion and extending radially-outward with respect to the optical axis; and
a visual marker positioned anterior to the cavity; and
a removable lens configured to removably couple with the base lens, the removable lens having
a lens portion; and
a tab extending radially-outward from the lens portion and configured to extend at least partially into the cavity when the removable lens is coupled to the base lens, wherein at least a portion of the tab is positioned posterior to the visual marker and at least partially hidden from view from an anterior-posterior perspective when the removable lens is coupled to the base lens.

2. The AIOL of claim 1 wherein the visual marker is a different color from at least a portion of base lens surrounding the visual marker.

3. The AIOL of claim 1 wherein the visual marker is opaque.

4. The AIOL of claim 1 wherein the visual marker is translucent

5. The AIOL of claim 1 wherein the visual marker is less transparent than at least a portion of the base lens surrounding the visual marker.

6. The AIOL of claim 1 wherein the visual marker comprises a darkened and/or colored silicone structure.

7. The AIOL of claim 6 wherein the visual marker is formed in a mold separate from the base lens.

8. The AIOL of claim 6 wherein the visual marker is partially cured and then co-molded with the base lens during manufacture.

9. The AIOL of claim 1 wherein the visual marker is adhered to the base lens during manufacture.

10. The AIOL of claim 1 wherein the visual marker is overmolded to the base lens during manufacture.

11. The AIOL of claim 1 wherein the tab is coplanar with the lens portion of the removable lens.

12. A method of assembling an accommodating intraocular lens (AIOL), the method comprising:
providing a base lens, the base lens having
an accommodating lens;
an optical axis passing through the accommodating lens;
a haptic portion positioned radially-outward from the accommodating lens with respect to the optical axis;
a cavity formed in the haptic portion and extending radially-outward with respect to the optical axis; and
a visual marker positioned anterior to the cavity
coupling a removable lens to the base lens by inserting a tab of the removable lens into the cavity such that the tab is positioned posterior of the visual marker and overlaps the marker in a direction parallel to the optical axis; and
aligning a lens portion of the removable lens with the optical axis.

13. The method of claim 12 wherein the removable lens is coupled to the base lens after the base lens is implanted in an eye of a patient.

14. The method of claim 12 wherein the visual marker hides a portion of the tab when observed from a position anterior to the visual marker.

15. The method of claim 12, further comprising overmolding the visual marker to the base lens.

16. The method of claim 12 wherein the visual marker is overmolded to the base lens.

17. The method of claim 12 wherein the visual marker is less transparent than at least a portion of the base lens surrounding the visual marker.

18. The method of claim 12 wherein the visual marker is opaque.

19. The method of claim 12, further comprising adhering the visual marker to the base lens.

20. The method of claim 12, further comprising co-molding the visual marker with the base lens.
